(19) United States
(12) Patent Application Publication
(10) Pub. No.: US 2015/0366620 A1
(43) Pub. Date: Dec. 24, 2015

(54) SYSTEM AND METHOD FOR USING GUIDE CLAMP FOR PORT BASED PROCEDURE

(71) Applicants: PIRON Cameron, Toronto (CA); WOOD Michael, Toronto (CA); SELA Gal, Toronto (CA); RICHMOND Joshua, Toronto (CA); YUWARAJ Murugathas, Toronto (CA); MCFAYDEN Stephen, Toronto (CA)

(72) Inventors: PIRON Cameron, Toronto (CA); WOOD Michael, Toronto (CA); SELA Gal, Toronto (CA); RICHMOND Joshua, Toronto (CA); YUWARAJ Murugathas, Toronto (CA); MCFAYDEN Stephen, Toronto (CA)

(21) Appl. No.: 14/398,855

(22) PCT Filed: Mar. 14, 2014
(86) PCT No.: PCT/CA2014/050257

§ 371 Date: Nov. 4, 2014

Related U.S. Application Data
(60) Provisional application No. 61/801,530, filed on Mar. 15, 2013.
(60) Provisional application No. 61/818,280, filed on May 1, 2013.

(51) Int. Cl. A61B 19/00; A61B 17/34

(52) U.S. Cl. CPC A61B 19/201; A61B 17/3421; A61B 19/26; A61B 19/5244; A61B 2019/208; A61B 2019/5287; A61B 2019/5259

(57) ABSTRACT

A guide for use with an access port for port-based surgery. The guide includes a body positionable over a surgical opening and a grip coupled to the body for removably receiving the access port into the surgical opening. At least one of the body and the grip is configured to restrict movement of the received access port to a limited range of motion.

### CROSS REFERENCE TO RELATED APPLICATIONS

The present disclosure claims priority from U.S. provisional patent applications Nos. 61/801,530 filed Mar. 15, 2013; and 61/818,280 filed May 1, 2013, the entireties of which are hereby incorporated by reference.

### FIELD

The present disclosure relates to navigation systems and devices and methods for minimally invasive therapy and image guided medical procedures.

### BACKGROUND

Minimally invasive neuro-surgical procedures typically require geometrically accurate and patient-registered imaging data to facilitate tissue differentiation and targeting. In typical procedures, however, optimal integration between imaging data (e.g., pre-surgical and intra-operative), surgical access, and resection devices remains lacking and the surgeon must cognitively integrate such information.

Pre-operative imaging data, such as Magnetic Resonance Imaging (MRI), Computerized Tomography (CT) and Positron Emission Tomography (PET), may conventionally be integrated into the surgical room statically through a viewing station, or dynamically through a navigation system. The navigation system may register devices to a patient, and a patient to the pre-operative scans, in order to allow for instruments to be tracked relative to the patient and viewed on a monitor in the context of the pre-operative information.

Intra-operative imaging systems may include microscopes, endoscopes, or external scopes. These are optical instruments that acquire optical wavelength imaging (e.g., 2D, or stereoscopic) at a higher resolution than what can be typically seen with the surgeon's unassisted eye. This optical information may be displayed during surgery on a screen for the surgeon to view as a video feed, while the navigated pre-operative imaging data (e.g., MRI, CT or PET data) typically may be presented on a separate screen.

During a port-based surgery, the surgical site of interest is typically accessed via an access port that serves to provide a path for surgical instruments to access the surgical site. However, there may be problems that preclude or impair the ability to perform port-based navigation in an intraoperative setting. For example, the position of the access port axis relative to a typical tracking device (TD) (e.g., an infrared camera) may be a free and uncontrolled parameter that negatively impacts the accurate determination of access port orientation. Furthermore, the presence of various equipment in the surgical area may limit the ability of the TD to track the access port. Also, the angular orientation of the access port may be adjusted by the surgeon in order to access different areas within the brain during a procedure. This change in orientation may make navigation of the access port more difficult and challenging. As well, the need for the surgeon to manually position and orient the access port, for example to hold the port at a desired position and orientation during surgery, typically leaves the surgeon with only one free hand to perform the actual surgery.

### SUMMARY

In some examples, the present disclosure provides a guide for use with an access port for port-based surgery, which may include: a body positionable over a surgical opening; and a grip coupled to the body for removably receiving the access port into the surgical opening; wherein at least one of the body and the grip is configured to restrict movement of the received access port to a limited range of motion.

In some examples, the present disclosure provides a system for port-based surgery, which may include the guide described above; and the access port for insertion into the surgical opening, the access port being receivable by the guide.

### BRIEF DESCRIPTION OF THE DRAWINGS

Reference will now be made, by way of example, to the accompanying drawings which show example embodiments of the present application, and in which:

FIG. 1 shows an example navigation system to support minimally invasive access port-based surgery;

FIG. 2 is a diagram illustrating system components of an example navigation system;

FIG. 3A is a flow chart illustrating an example method involved in a surgical procedure using the example navigation system of FIG. 2;

FIG. 3B is a flow chart illustrating an example method of registering a patient for a surgical procedure as outlined in FIG. 3A;

FIG. 4 shows an example system for tracking an access port in a port-based surgical procedure;

FIG. 5 shows a block diagram of an example system configuration, including a control and processing unit and external components;

FIGS. 6A and 6B illustrate an example embodiment of a guide for an access port, and additional structure to hold the guide body;

FIG. 6C shows an example embodiment of a guide for an access port, including a locking mechanism;

FIG. 6D shows different views of an example arrangement for providing fiducial markers for an access port; and

FIGS. 7A-7D are diagrams illustrating an example embodiment of a self-centering guide for an access port.

Similar reference numerals may have been used in different figures to denote similar components.

### DESCRIPTION OF EXAMPLE EMBODIMENTS

The systems and methods described herein may be useful in the field of neurosurgery, including oncological care, neurodegenerative disease, stroke, brain trauma and orthopedic surgery. Persons of skill will appreciate the ability to extend these concepts to other conditions or fields of medicine. It should be noted that the present disclosure may be applicable to surgical procedures for brain, spine, knee and any other region of the body that may use an access port or small orifice to access the interior of the human body.

Various example apparatuses or processes will be described below. No example embodiment described below limits any claimed embodiment and any claimed embodiments may cover processes or apparatuses that differ from those examples described below. The claimed embodiments are not limited to apparatuses or processes having all of the features of any one apparatus or process described below or to features common to multiple or all of the apparatuses or processes described below. It is possible that an apparatus or process described below is not an embodiment of any claimed embodiment.

Furthermore, numerous specific details are set forth in order to provide a thorough understanding of the disclosure. However, it will be understood by those of ordinary skill in the art that the embodiments described herein may be practiced without these specific details. In other instances, well-known methods, procedures and components have not been described in detail so as not to obscure the embodiments described herein.

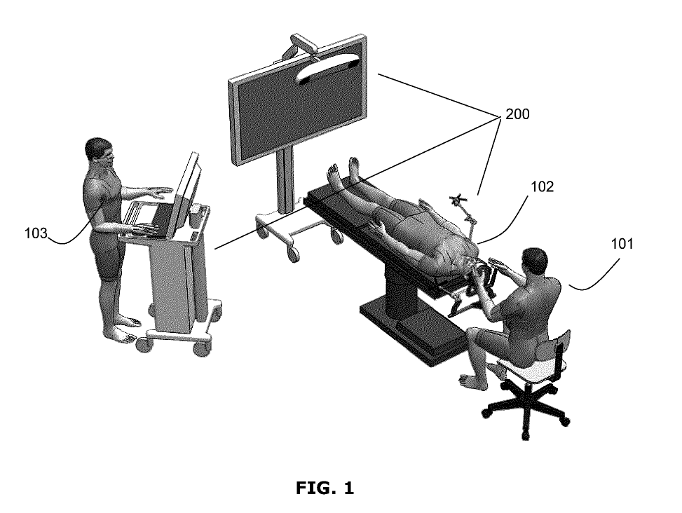

FIG. 1 illustrates a perspective view of an example minimally invasive port-based surgical procedure. As shown in FIG. 1, a surgeon 101 may conduct a minimally invasive port-based surgery on a patient 102 in an operating room (OR) environment. A localization or navigation system 200 (described further below) may be used to assist the surgeon 101 during the procedure. Optionally, an operator 103 may be present to operate, control and provide assistance with the navigation system 200.

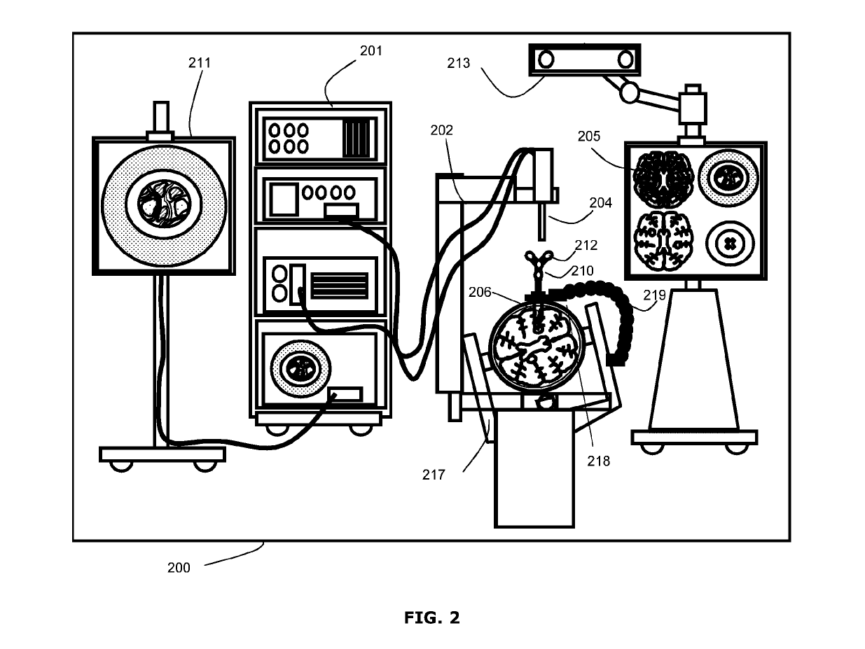

FIG. 2 shows a diagram illustrating components of an example medical navigation system 200. The medical navigation system 200 illustrates the context in which a guide for an access port, such as that described herein, may be used. The medical navigation system 200 may include one or more displays 205, 211 for displaying a video image, an equipment tower 201, and a mechanical arm 202, which may support an optical scope 204 (which may also be referred to as an external scope). The equipment tower 201 may be mounted on a frame (e.g., a rack or cart) and may contain a power supply and a computer or controller that may execute planning software, navigation software and/or other software to manage the mechanical arm 202 and tracked instruments. In some examples, the equipment tower 201 may be a single tower configuration operating with dual displays 211, 205, however other configurations may also exist (e.g., dual tower, single display, etc.). Furthermore, the equipment tower 201 may also be configured with a universal power supply (UPS) to provide for emergency power, in addition to a regular AC adapter power supply.

A portion of the patient's anatomy may be held in place by a holder. For example, as shown the patient's head and brain may be held in place by a head holder 217. An access port 206 and associated introducer 210 may be inserted into the head, to provide access to a surgical site in the head. In FIG. 2, fiducial markers 212 may be connected to the introducer 210 for tracking by the tracking camera 213, which may provide positional information of the introducer 210 from the navigation system 200. In some examples, the fiducial markers 212 may be alternatively or additionally attached to access port 206. In some examples, the tracking camera 213 may be a 3D infrared optical tracking stereo camera similar to one made by Northern Digital Imaging (NDI). In some examples, the tracking camera 213 may be a magnetic camera, such as a field transmitter that may use receiver coils as fiducial markers. Location data of the mechanical arm 202 and/or access port 206 may be determined by the tracking camera 213 by detection of the fiducial markers 212 placed on or otherwise in fixed relation (e.g., in rigid connection) to any of the mechanical arm 202, the access port 206, the introducer 210, and/or other pointing tools. The fiducial marker(s) 212 may be active or passive markers. The secondary display 205 may provide output of the computed data of the navigation system 200. In some examples, the output provided by the secondary display 205 may include axial, sagittal and coronal views of patient anatomy as part of a multi-view output.

Minimally invasive brain surgery using an access port 206 is a method of performing surgery on brain tumors. In order to introduce an access port 206 into the brain, the introducer 210, having an atraumatic tip, may be positioned within the access port 206 and employed to position the access port 206 within the patient's brain. The introducer 210 may include fiducial markers 212 for tracking position and orientation of the introducer 210. The fiducial markers 212 may be passive (e.g., reflective spheres for use with an optical tracking system, or pick-up coils for use with an electromagnetic tracking system). The fiducial markers 212 may be detected by the tracking camera 213 and the respective positions of the tracked tool may be inferred by tracking software executed by a computer or controller in connection with the navigation system 200.

Once the access port 206 has been positioned into the brain, the associated introducer 210 may be removed to allow for access to the surgical site of interest, through the central opening of the access port 206. In the present disclosure, tracking of the access port 206 may be provided by an access port guide or by attaching markers to the access port 206 itself, as described further below.

As shown in FIG. 2, a guide clamp 218 (or more generally a guide) for holding the access port 206 may be provided. The guide clamp 218 can optionally engage and disengage with the access port 206 without needing to remove the access port 206 from the patient. In some examples, the access port 206 may be moveable relative to the guide clamp 218, while in the guide clamp 218. For example, the access port 206 may be able to slide up and down (e.g., along the longitudinal axis of the access port 206) relative to the guide clamp 218 while the guide clamp 218 is in a closed position. A locking mechanism may be attached to or integrated with the guide clamp 218, and may optionally be actuatable with one hand, as described further below.

An articulated arm 219 may be provided to hold the guide clamp 218. The articulated arm 219 may have up to six degrees of freedom to position the guide clamp 218. The articulated arm 219 may be lockable to fix its position and orientation, once a desired position is achieved. The articulated arm 219 may be attached or attachable to a point based on the patient head holder 217, or another suitable point (e.g., on another patient support, such as on the surgical bed), to ensure that when locked in place, the guide clamp 218 does not move relative to the patient's head.

In a surgical operating room (or theatre), setup of a navigation system may be relatively complicated; there may be many pieces of equipment associated with the surgical procedure, as well as elements of the navigation system 200. Further, setup time typically increases as more equipment is added. To assist in addressing this, the navigation system 200 may include two additional wide-field cameras to enable video overlay information. One wide-field camera may be mounted on the optical scope 204, and a second wide-field camera may be mounted on the tracking camera 213. Video overlay information can then be inserted into displayed images, where the overlay information may illustrate the physical space where accuracy of the 3D tracking system (which is typically part of the navigation system) is greater, may illustrate the available range of motion of the mechanical arm 202 and/or the optical scope 204, and/or may help to guide head and/or patient positioning.

The navigation system 200 may provide tools to the neurosurgeon that may help to provide more relevant information to the surgeon, and may assist in improving performance and accuracy of port-based neurosurgical operations. Although described in the present disclosure in the context of port-based neurosurgery (e.g., for removal of brain tumors and/or for treatment of intracranial hemorrhages (ICH)), the navigation system 200 may also be suitable for one or more of: brain biopsy, functional/deep-brain stimulation, catheter/shunt placement (in the brain or elsewhere), open craniotomies, and/or endonasal/skull-based/ear-nose-throat (ENT) procedures, among others. The same navigation system 200 may be used for carrying out any or all of these procedures, with or without modification as appropriate.

For example, although the present disclosure may discuss the navigation system 200 in the context of neurosurgery, the same navigation system 200 may be used to carry out a diagnostic procedure, such as brain biopsy. A brain biopsy may involve the insertion of a thin needle into a patient's brain for purposes of removing a sample of brain tissue. The brain tissue may be subsequently assessed by a pathologist to determine if it is cancerous, for example. Brain biopsy procedures may be conducted with or without a stereotactic frame. Both types of procedures may be performed using image-guidance. Frameless biopsies, in particular, may be conducted using the navigation system 200.

In some examples, the tracking camera 213 may be part of any suitable tracking system. In some examples, the tracking camera 213 (and any associated tracking system that uses the tracking camera 213) may be replaced with any suitable tracking system which may or may not use camera-based tracking techniques. For example, a tracking system that does not use the tracking camera 213, such as a radiofrequency tracking system, may be used with the navigation system 200.

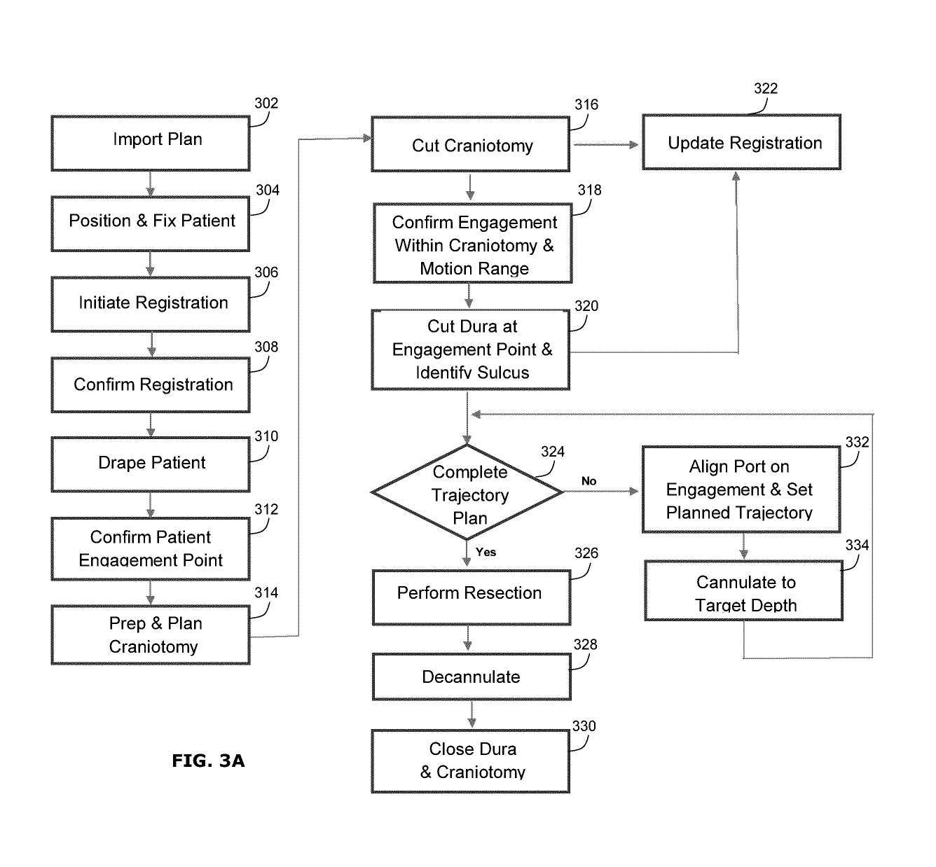

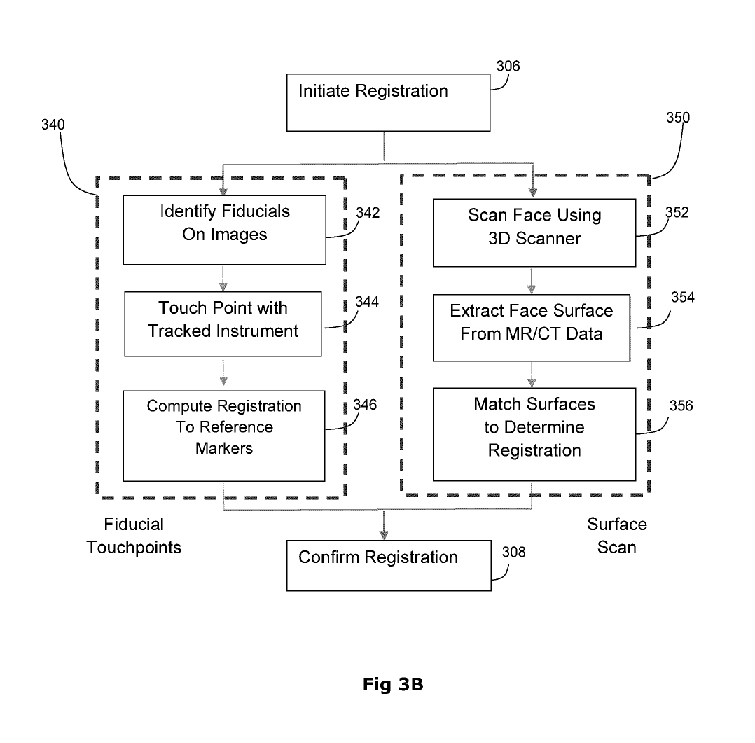

FIG. 3A is a flow chart illustrating an example method 300 of performing a port-based surgical procedure using a navigation system, such as the medical navigation system 200 described above. At a first block 302, the port-based surgical plan may be imported. A detailed description of an example process to create and select a surgical plan is outlined in U.S. patent application Ser. No. ______, titled “PLANNING, NAVIGATION AND SIMULATION SYSTEMS AND METHODS FOR MINIMALLY INVASIVE THERAPY”, which claims priority from U.S. provisional patent application Nos. 61/800,155 and 61/924,993. The entireties of all these disclosures are incorporated herein by reference.

An example surgical plan may include pre-operative 3D imaging data (e.g., MRI, CT, PET or ultrasound data). The plan may include overlaid data, such as additional received inputs (e.g., sulci entry points, target locations, surgical outcome criteria and/or additional 3D image data information). The plan may also include a display of one or more planned trajectory paths (e.g., based on calculated score for a projected surgical path). Other surgical plans and/or methods may additionally or alternatively be used as inputs into the navigation system.

Once the plan has been imported into the navigation system at the block 302, the patient may be affixed into position (e.g., using a body holding mechanism, such as the head holder 217). The patient's head position may be also confirmed with the plan using appropriate navigation software (block 304), which in an example may be implemented by the computer or controller forming part of the equipment tower 201.

Next, registration of the patient may be initiated (block 306). The phrase “registration” may refer to the process of transforming different sets of data into one coordinate system. Data may include multiple photographs, data from different sensors, times, depths, or viewpoints, for example. The process of registration may be used in the context of the present disclosure for medical imaging, in which images from different imaging modalities may be co-registered. Registration may be used in order to be able to compare and/or integrate the data obtained from these different modalities.

Registration of the patient to a base reference frame may occur in various suitable ways. Example methods for registration may include:

Identification of features (natural or engineered) in the image data (e.g., MR and CT images) and indication of those same features on the actual patient using a pointer tool that may be tracked by the tracking camera;

Tracing a line on the curved profile of the patient's face or forehead with a pointer tool that may be tracked by the tracking camera, and matching this curved profile to the image data (e.g., 3D MR or CT volume);

Application of a tool of known geometry to the patient's face, where the tool may have targets tracked by the tracking camera; or

Using a surface acquisition tool (e.g., based on structured light) to extract a surface of the patient's face or forehead and matching the extracted surface to the image data (e.g., 3D MR or CT volume) using appropriate techniques.

Various registration techniques available to those skilled in the art may be suitable, and one or more of these techniques may be applied to the present disclosure. Non-limiting examples include intensity-based methods that compare intensity patterns in images via correlation metrics, as well as feature-based methods that find correspondence between image features such as points, lines, and contours, among other possible methods. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in MRI and PET. In the present disclosure, multi-modality registration methods may be used in medical imaging of the head and/or brain as images of a patient are frequently obtained from different scanners. Examples include registration of brain CT/MRI images or PET/CT images for tumor localization, registration of contrast-enhanced CT images against non-contrast-enhanced CT images, and registration of ultrasound and CT.

FIG. 3B shows a flow chart illustrating example methods that may be used to carry out the registration of the block 306. Block 340 illustrates an approach using fiducial touch points, while block 350 illustrates an approach using a surface scan. The block 350 is not typically used when fiducial touch points or a fiducial pointer is used.

If the use of fiducial touch points (block 340) is contemplated, the method may involve first identifying fiducial points on images (block 342), then touching the corresponding touch points on the patient with a tracked instrument (block 344). Next, the navigation system may compute the registration to reference markers (block 346).

If a surface scan procedure (block 350) is used, the patient's face may be scanned using a 3D scanner (block 352). Next, the face surface may be extracted from image data (e.g., MR or CT data) (block 354). Finally, the scanned surface and the extracted surface may be matched to each other to determine registration data points (block 356).

Upon completion of either the fiducial touch points (block 340) or surface scan (block 350) procedures, the data extracted may be computed and used to confirm registration at block 308, shown in FIG. 3A.

Referring back to FIG. 3A, once registration is confirmed (block 308), the patient may be draped (block 310). Typically, draping involves covering the patient and surrounding areas with a sterile barrier to create and maintain a sterile field during the surgical procedure. Draping may be used to eliminate the passage of microorganisms (e.g., bacteria) between non-sterile and sterile areas.

Upon completion of draping (block 310), the patient engagement points may be confirmed (block 312) and then the craniotomy may be prepared and planned (block 314).

Upon completion of the preparation and planning of the craniotomy (block 314), the craniotomy may be cut and a bone flap may be temporarily removed from the skull to access the brain (block 316). Registration data may be updated with the navigation system at this point (block 322).

Next, the engagement within craniotomy and the motion range may be confirmed (block 318). Next, the procedure may advance to cutting the dura at the engagement points and identifying the sulcus (block 320). Registration data may again be updated with the navigation system at this point (block 322).

In some examples, by focusing the camera's view on the surgical area of interest, update of the registration data (block 322) may be adjusted to help achieve a better match for the region of interest, while ignoring any non-uniform tissue deformation, for example, affecting areas outside of the region of interest. Additionally, by matching image overlay representations of tissue with an actual view of the tissue of interest, the particular tissue representation may be matched to the live video image, which may help to improve registration of the tissue of interest. For example, the registration may enable: matching a live video of the post craniotomy brain (with the brain exposed) with an imaged sulcal map; matching the position of exposed vessels in a live video with image segmentation of vessels; matching the position of lesion or tumor in a live video with image segmentation of the lesion and/or tumor; and/or matching a video image from endoscopy up the nasal cavity with bone rendering of bone surface on nasal cavity for endonasal alignment.

In some examples, multiple cameras can be used and overlaid with tracked instrument(s) views, which may allow multiple views of the image data and overlays to be presented at the same time. This may help to provide greater confidence in registration, or may enable easier detection of registration errors and their subsequent correction.

Thereafter, the cannulation process may be initiated. Cannulation typically involves inserting an access port into the brain, typically along a sulcus path as identified at 320, along a trajectory plan. Cannulation is typically an iterative process that may involve repeating the steps of aligning the port on engagement and setting the planned trajectory (block 332) and then cannulating to the target depth (block 334) until the complete trajectory plan is executed (block 324)

In some examples, the cannulation process may also support multi-point trajectories where a target (e.g., a tumor) may be accessed by cannulating to intermediate points, then adjusting the cannulation angle to get to the next point in a planned trajectory. This multi-point trajectory may be contrasted with straight-line trajectories where the target may be accessed by cannulating along a straight path directly towards the target. The multi-point trajectory may allow a cannulation trajectory to skirt around tissue that the surgeon may want to preserve. Navigating multi-point trajectories may be accomplished by physically reorienting (e.g., adjusting the angle of) a straight access port at different points along a planned path, or by using a flexible port, such as an access port with manipulatable bends that may be bent along the multi-point trajectory.

Once cannulation of the access port is complete, the surgeon may perform resection (block 326) to remove part of the brain and/or tumor of interest, with or without having first removed the introducer (if used). The surgeon may then decannulate (block 328) by removing the port from the brain. Finally, the surgeon may close the dura and complete the craniotomy (block 330). Some aspects of FIGS. 3A and 3B may be specific to port-based surgery, such as portions of blocks 328, 320, and 334. Appropriate portions of these blocks may be skipped or suitably modified when performing non-port-based surgery.

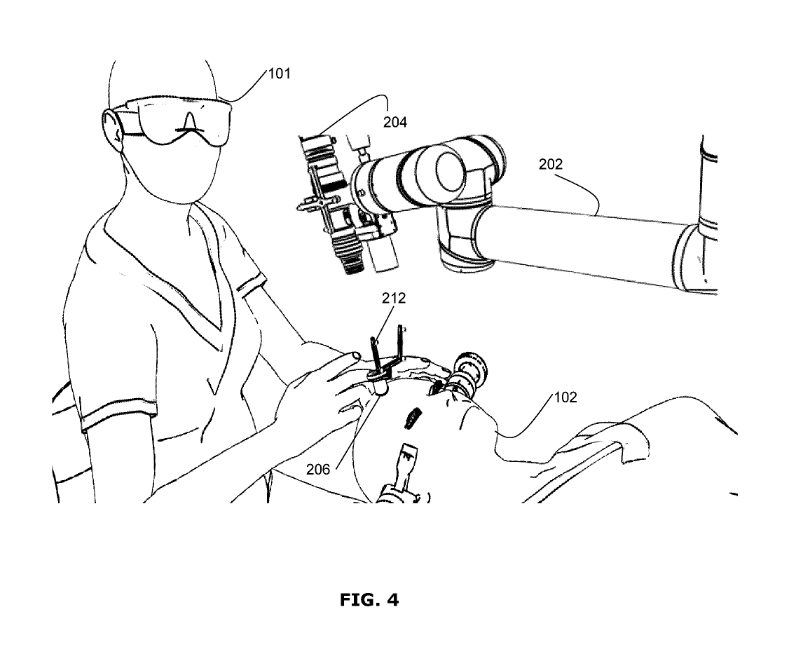

FIG. 4 illustrates certain components of a navigation system, in particular for tracking of tools in a port-based surgical procedure. In FIG. 4, the surgeon 101 may be in the process of performing neurosurgery, for example the surgeon 101 may be resecting a tumor out of the brain of the patient 102 through the access port 206. As discussed above, the optical scope 204 may be attached to a mechanical arm 202, and may be used to view down the access port 206 at a sufficient magnification to allow for enhanced visibility down the access port 206. The output of the optical scope 204 may be received by one or more computers or controllers to generate a view that may be depicted on a visual display (e.g., one or more monitors).

The active or passive fiducial markers 212 may be placed on tools (e.g., the access port 206 and/or the optical scope 204) to be tracked, to determine the location and orientation of these tools using the tracking camera and navigation system. The markers 212 may be captured by a stereo camera of the tracking system to give identifiable points for tracking the tools. A tracked tool may be defined by a grouping of markers 212, which may define a rigid body to the tracking system. This may in turn be used to determine the position and orientation in 3D of a tracked tool in a virtual space. In typical use with navigation systems, at least three markers 212 are provided on a tracked tool to define the tool in virtual space, however it is known to be advantageous for four or more markers 212 to be used.

Camera images capturing the markers 212 may be logged and tracked, by, for example, a closed circuit television (CCTV) camera. The markers 212 may be selected to enable or assist in segmentation in the captured images. For example, infrared (IR)-reflecting markers and an IR light source from the direction of the camera may be used. An example of such an apparatus may be tracking devices such as the Polaris® system available from Northern Digital Inc. In some examples, the spatial position of the tracked tool and/or the actual and desired position of the mechanical arm 202 may be determined by optical detection using a camera. The optical detection may be done using an optical camera, rendering the markers 212 optically visible.

In some examples, the markers 212 (e.g., reflectospheres) may be used in combination with a suitable tracking system, to determine the spatial positioning position of the tracked tools within the operating theatre. Different tools and/or targets may be provided with respect to sets of markers 212 in different configurations. Differentiation of the different tools and/or targets and their corresponding virtual volumes may be possible based on the specification configuration and/or orientation of the different sets of markers 212 relative to one another, enabling each such tool and/or target to have a distinct individual identity within the navigation system. The individual identifiers may provide information to the system, such as information relating to the size and/or shape of the tool within the system. The identifier may also provide additional information such as the tool's central point or the tool's central axis, among other information. The virtual tool may also be determinable from a database of tools stored in or provided to the navigation system. The markers 212 may be tracked relative to a reference point or reference object in the operating room, such as the patient.

Various types of markers may be used. The markers 212 may all be the same type or may include a combination of two or more different types. Possible types of markers that could be used may include reflective markers, radiofrequency (RF) markers, electromagnetic (EM) markers, pulsed or un-pulsed light-emitting diode (LED) markers, glass markers, reflective adhesives, or reflective unique structures or patterns, among others. RF and EM markers may have specific signatures for the specific tools they may be attached to. Reflective adhesives, structures and patterns, glass markers, and LED markers may be detectable using optical detectors, while RF and EM markers may be detectable using antennas. Different marker types may be selected to suit different operating conditions. For example, using EM and RF markers may enable tracking of tools without requiring a line-of-sight from a tracking camera to the markers 212, and using an optical tracking system may avoid additional noise from electrical emission and detection systems.

In some examples, the markers 212 may include printed or 3D designs that may be used for detection by an auxiliary camera, such as a wide-field camera (not shown) and/or the optical scope 204. Printed markers may also be used as a calibration pattern, for example to provide distance information (e.g., 3D distance information) to an optical detector. Printed identification markers may include designs such as concentric circles with different ring spacing and/or different types of bar codes, among other designs. In some examples, in addition to or in place of using markers 212, the contours of known objects (e.g., the side of the access port 206) could be captured by and identified using optical imaging devices and the tracking system.

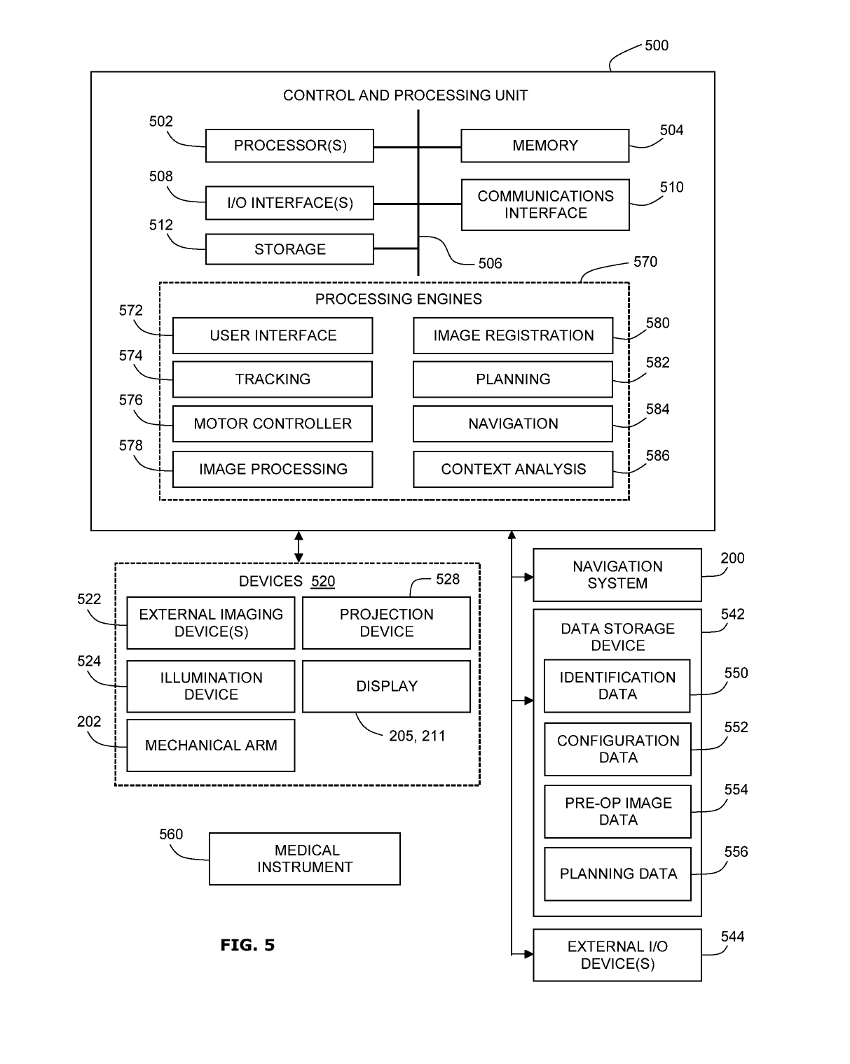

FIG. 5 shows a block diagram of an example system configuration that may be used to carry out the functions of a navigation system, as disclosed herein. The example system may include a control and processing unit 500 and other external components.

In some examples, the control and processing unit 500 may include one or more processors 502 (for example, a CPU and/or microprocessor), one or more memories 504 (which may include random access memory (RAM) and/or read-only memory (ROM)), a system bus 506, one or more input/output interfaces 508 (such as a user interface for a user (e.g., a clinician or a surgeon) to provide various inputs (e.g., to perform trajectory planning or run simulations)), one or more communications interfaces 510, and one or more internal storage devices 512 (e.g. a hard disk drive, compact disk drive and/or internal flash memory). The control and processing unit may also include a power supply (not shown).

The control and processing unit 500 may interface with one or more other external devices, such as a tracking system or navigation system (e.g., the navigation system 200 of FIG. 2), a data storage device 542, and external input and/or output devices 544 which may include, for example, one or more of a display, keyboard, mouse, foot pedal, microphone and speaker. The data storage device 542 may include any one or more suitable data storage devices, such as a local or remote computing device (e.g., a computer, a hard drive, a digital media device, or a server) which may have a database stored thereon. In the example shown in FIG. 5, the data storage device 542 may store identification data 550 for identifying one or more medical instruments 560 and configuration data 552 that may associate customized configuration parameters with the one or more medical instruments 560. The data storage device 542 may also store preoperative image data 554 and/or medical procedure planning data 556. Although the data storage device 542 is shown as a single device, the data storage device 542 may be provided as one or more storage devices.

The medical instrument(s) 560 may be identifiable by the control and processing unit 500. The medical instrument(s) 560 may be connected to, and controlled by, the control and processing unit 500, or may be operated or otherwise employed independently of the control and processing unit 500. The navigation system 200 may be employed to track one or more of the medical instrument(s) 560 and spatially register the one or more tracked medical instruments 560 to an intraoperative reference frame, for example as discussed above.

The control and processing unit 500 may also interface with one or more other configurable devices 520, and may intraoperatively reconfigure one or more of such device(s) 520 based on configuration parameters obtained from configuration data 552, for example. Examples of the device(s) 520 may include one or more external imaging devices 522, one or more illumination devices 524, the mechanical arm 202, one or more projection devices 528, and one or more displays 205, 211.

Various embodiments and aspects of the present disclosure may be implemented via the processor(s) 502 and/or memory(ies) 504. For example, one or more of the functionalities and methods described herein may be at least partially implemented via hardware logic in the processor(s) 502 and/or at least partially using instructions stored in the memory(ies) 504, as one or more processing engines 570 (also referred to as modules). Example processing engines 570 include, but are not limited to, a user interface engine 572, a tracking engine 574, a motor controller 576, an image processing engine 578, an image registration engine 580, a procedure planning engine 582, a navigation engine 584, and a context analysis engine 586. Although certain engines (or modules) are described, it should be understood that engines or modules need not be specifically defined in the instructions, and an engine or module may be used to implement any combination of functions.

It is to be understood that the system is not intended to be limited to the components shown in FIG. 5. For example, one or more components of the control and processing unit 500 may be provided as an external component or device. Although only one of each component is illustrated in FIG. 5, any number of each component can be included. For example, a computer typically contains a number of different data storage media. Furthermore, although the bus 506 is depicted as a single connection between all of the components, the bus 506 may represent one or more circuits, devices or communication channels which link two or more of the components. For example, in personal computers, the bus 506 may include or may be a motherboard.

In some examples, the navigation engine 584 may be provided as an external navigation system that may interface with or be integrated with the control and processing unit 500.

Some embodiments or aspects of the present disclosure may be implemented using the processor 502 without additional instructions stored in the memory 504. Some embodiments or aspects of the present disclosure may be implemented using instructions stored in the memory 504 for execution by one or more general purpose microprocessors. In some examples, the control and processing unit 500 (which may be also referred to as a computer control system) may be, or may include, a general purpose computer or any other hardware equivalents configured for operation in space. The control and processing unit 500 may also be implemented as one or more physical devices that may be coupled to the processor(s) 502 through one or more communications channels or interfaces. For example, the control and processing unit 500 may be implemented using application specific integrated circuits (ASIC). In some examples, the control and processing unit 500 may be implemented as a combination of hardware and software, such as where the software may be loaded into the processor(s) 502 from the memory(ies) 504 or internal storage(s) 512, or from an external source (e.g., via the communication interface(s) 510, such as over a network connection). Thus, the present disclosure is not limited to a specific configuration of hardware and/or software.

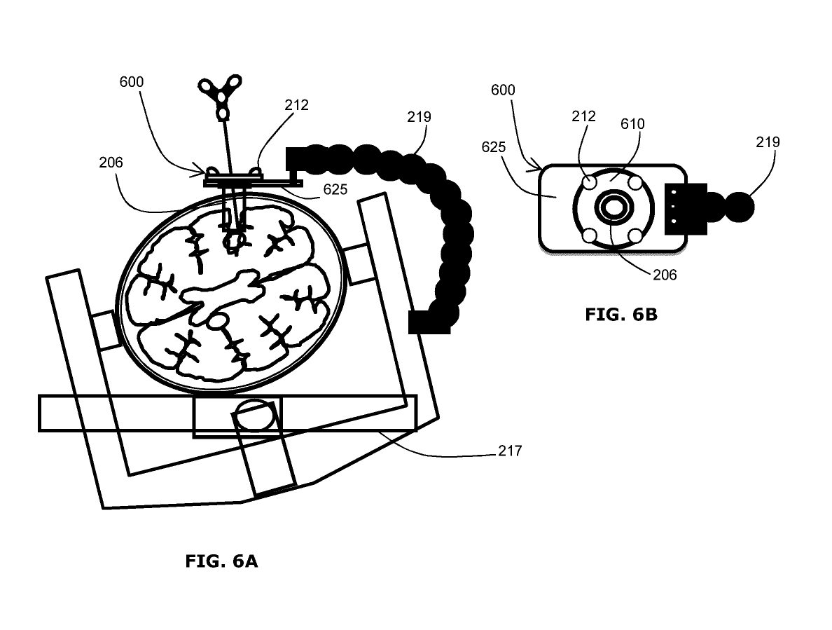

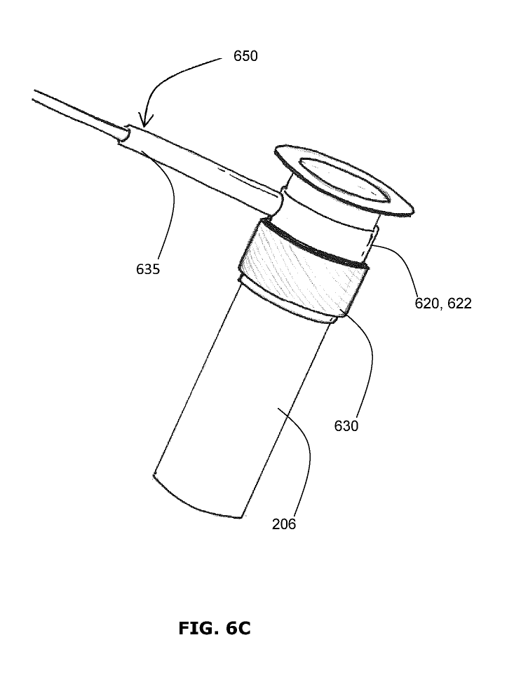

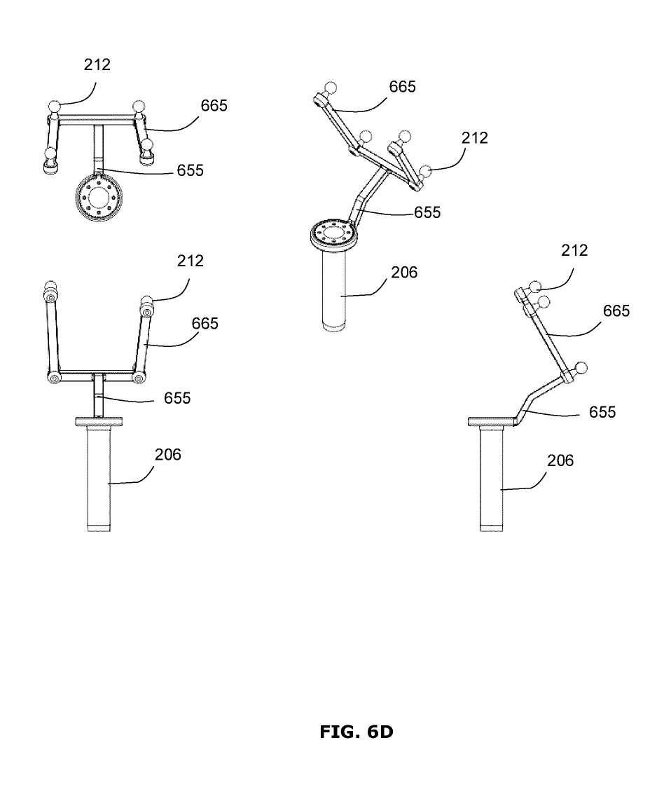

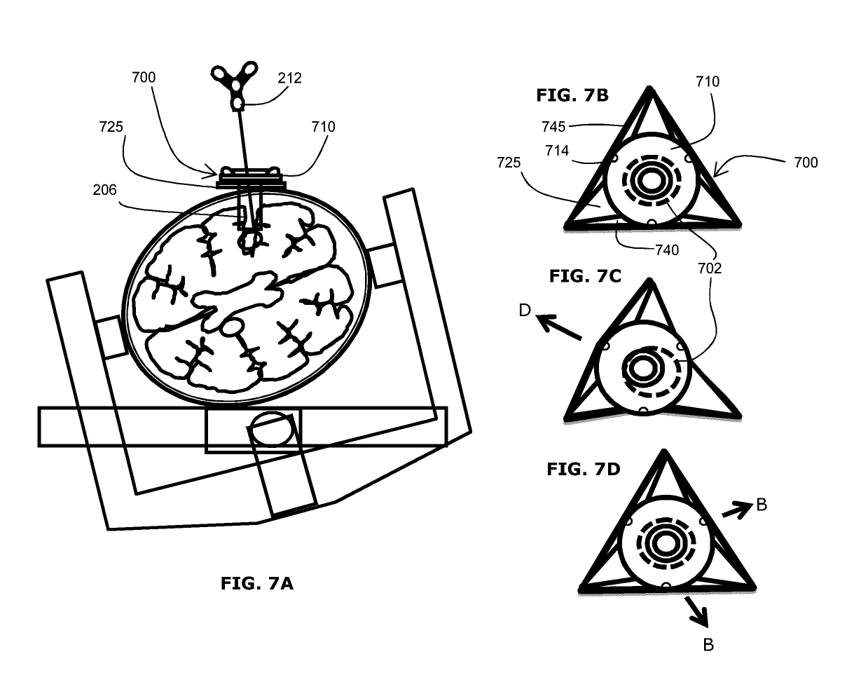

As discussed earlier in the present disclosure, placement and manipulation of the access port 206 may be assisted by use of a guide. Example embodiments of such a guide are now discussed, with reference to FIGS. 6A-6D and 7A-7D. The guide may also be referred to as a guide clamp (e.g., in example embodiments where the guide includes a clamp). The access port 206 may have a fixed position and orientation with respect to the guide, or the access port 206 may have a limited range of motion with respect to the guide, as discussed below. The guide may be adjustable to permit variation between a fixed position and/or orientation of the access port 206 and a limited range of motion for the access port 206. Use of the guide may help to track the position and orientation of the access port 206, for example where the guide itself is tracked by a navigation system 200; or where the guide has a known fixed position and orientation with respect to the articulated arm 219 holding the guide, and the position and orientation of the articulated arm 219 is known (e.g., the articulated arm 219 is tracked by the tracking system or the articulated arm 219 includes joint encoders that log movement of the articulated arm 219).

In some examples, such as the example shown in FIG. 6D, the access port 206 may be provided with fiducial markers 212. For example, a tracking arm 655 may be removably or permanently attached to the access port 206, and the fiducial markers 212 may be provided on such tracking arm 655. The tracking arm 655 may be attached at or near the portion of the access port 206 that is intended to remain outside of the surgical opening, and the tracking arm 655 may be configured to extend away from and not occlude the opening of the access port 206, to avoid interfering with viewing through the access port 206, and to avoid interfering with surgical tools that may be inserted through the access port 206. The tracking arm 655 may be substantially rigid and may have a known and fixed position and orientation with respect to the access port 206. The tracking arm 655 may include one or more branches 665 on which markers 212 may be provided. The branches 665 may preferably be non-uniform and/or non-symmetrical (e.g., having unequal lengths), in order to enable the tracking software to unambiguously determine the position, orientation, and identity of the access port 206 in the navigation system. Such an arrangement may enable clear visibility of the markers 212 to the tracking camera while avoiding interference with the surgical tools.

In some examples, the markers 212 may additionally or alternatively be provided on one or more of probes, introducers and/or guides, for example by providing such components with a tracking arm 655 as described above.

FIGS. 6A and 6C illustrate an example embodiment of a guide 600 that may be removably attachable to the articulated arm 219. FIG. 6A shows a side view of the guide clamp assembly, and 6B shows a top view of the guide clamp assembly.

In FIG. 6A, when attached to the articulated arm 219, the guide 600 may be moveable or may be fixed in position and/or orientation with respect to the articulated arm 219. The guide 600 may include a body 625 providing a grip 610 for holding the access port 206. Markers are shown attached to grip 610 for the purposes of tracking the grip 610 with a navigation system 200. In an alternate embodiment, markers 212 may be provided on the body 625 of the guide 600.

The access port 206 may be removably received by the grip 610, even when the access port 206 has been partially or fully introduced into the patient. For example, the grip 610 may be configured to engage and disengage with the access port 206 without requiring the access port 206 to be removed from the patient. The grip 610 may be opened to enable receipt or removal of the access port 206, and may be closed to retain the access port 206 within the grip 610. In an embodiment, the grip 610 may be opened and closed about a hinge (not shown). In some examples, the access port 206, when received in the grip 610, may be moveable within a limited range of motion. For example, the access port 206 may be slideable up and down along its longitudinal axis while received in the grip 610. In some examples, the access port 206 may be fixed in position and orientation relative to the grip 610 when received in the grip 610. In some examples, the grip 610 may be adjusted between locked and unlocked configurations, wherein which the access port 206 in the unlocked configuration may have more freedom of motion (e.g., more freedom to change position and/or orientation) than when held by the grip 610 in the locked configuration (e.g., less or no freedom to change position and/or orientation). The grip 610 may therefore be adjustable to varying amounts between the locked and unlocked configurations, to enable different amounts of freedom of motion for the access port 206.

In some examples, the grip 610 may have a textured or high-friction surface to provide for better gripping and manipulation of the access port 206. In a further embodiment, the grip 610 may consist of two (or more) adjustable claws.

In a further embodiment, the grip 610 may be connected to the body 625 in a manner that is elastomeric in nature, so that the grip 610 may slide relative to the boy 625 and return to a neutral position when force is no longer being applied to deflect the grip 610. This embodiment allows movement of the access port 206 to be controlled and constrained (above a fixed plane) and therefore may provide the surgeon with improved control over the movement of the access port 206 within the brain.

FIG. 6C shows an example guide 650 in which a grip 620 may be provided within a body, such as a grip holder 622. The grip 620 may be adjusted using a locking mechanism provided by the grip holder 622, such as a rotatable collar 630, to vary the amount of freedom of motion available to the received access port 206. The locking mechanism may be actuatable using one hand, which may enable a surgeon to vary between locked and unlocked configurations of the grip holder 622 while holding a surgical tool or the access port 206 in the other hand. For example, the rotatable collar 630 may engage the grip holder 622, such that rotating or twisting the collar 630 in one direction will cause the grip holder 622 (and hence the grip 620) to frictionally engage or lock onto the received access port 206, and disengage or loosen away from the received access port 206 when rotated or twisted in the opposite direction. In a further embodiment, the rotatable collar 630 may be threaded upon a series of threaded axial fingers (not shown) in the grip holder 622, such that rotation of the rotatable collar in one sense causes the grip 620 to be inwardly directed and create a friction fit between the grip 620 and the access port 206. When the rotatable collar 630 causes the grip holder 622 to be locked, the access port 206 may be fixed in position and orientation with respect to the guide 650; when the collar 630 causes the grip holder 622 to be loosened or unlocked, the access port 206 may be moveable along its longitudinal axis with respect to the guide 650 to permit the access port 206 to be inserted into or withdrawn from the patient's brain.

The grip holder 622 may include a linkage 635 for attaching the guide 650 to the articulated arm 219. The linkage 635 may be flexible or rigid, or may be adjustable between flexible and rigid. Flexibility of the linkage 635 may be useful to enable movement of the received access port 206, relative to the brain, in order to better access various locations within the brain, while rigidity of the linkage 635 may be useful to help ensure that the received access port 206 remains substantially fixed in place relative to the head holder 217 or other object or tool to which the guide 650 is connected. The linkage 635 may have resilient or biasing properties, such that the linkage 635 may allow for limited freedom of movement of the received access port 206, but may tend to return the access port 206 to a centered or neutral position and orientation. Examples of resilient properties include the ability of the linkage 635 to elastically deform in any direction so that the linkage 635 returns to its neutral state after deformation. The linkage 635 may also exhibit this behaviour along the longitudinal axis using a spring loaded telescopic mechanism, for example.

For example, the linkage 635 may be an elongate member, such as a slender bar or rod. When the access port 206 is moved to various positions, the linkage 635 may oppose such movement, and may return the access port back to the centered position. The flexibility and rigidity of the linkage 635 may be manually adjusted (e.g., using one hand). In some examples, the grip holder 622 may be mechanically coupled to the linkage 635 such that rotation of the collar 630 may also serve to adjust the orientation and/or flexibility of the grip holder 622 in relation to the linkage 635.

In some examples, the grip 610, 620 may comprise separate pieces (not shown) that may be assembled together to hold the access port 206. For example, the grip 610, 620 may have a first portion that is attached to the body 625, 622 of the guide 600, 650 and a second portion that can be attached to the first portion to hold the access port 206 between the first and second portions. Where the grip 610, 620 is configured as a clamp, the first and second portions may be halves of a clamp, for example they may be U-shaped pieces having inner surfaces complementary to the outside surface of the access port 206. The second portion may be attached to and locked onto the first portion, for example using clasps, to lock the access port 206 in the grip 610, 620. In some examples, the second portion may be hingedly attached to the first portion and may swing open to permit the access port 206 to be positioned in the body 625, 622 and the second portion may swing close to hold the access port 206 in the body 625, 622. A locking mechanism, such as the rotatable collar 630, may be used to attach the second portion to the first portion and adjust how tightly the first and second portions clamp onto the access port 206.

FIGS. 7A-7D show another example embodiment of a guide 700 that may be used with the access port 206. The guide 700 in this example need not be attached to an articulated arm 219, as the guide 700 is generally configured to be positioned directly over the surgical opening (e.g., a burr-hole in the patient's head). The example guide 700 may provide better surgical access to the patient's anatomy, compared to the example guide 600 discussed above, and may have less stringent requirements for immobilizing the patient's head. In the example shown, markers 212 may be provided on a separate instrument (e.g., a separate pointing tool), however in other examples markers 212 may be provided on the guide 700.

The guide 700 may include a body 725, which is configured to be in contact with a patient's head, and which optionally may be shaped or be shapable to accommodate or conform to the curvature of a patient's head (not shown). In the example shown, the body 725 may have a substantially triangular shape, however other configurations, such as cruciform, hub and spoke, or other circular or square shapes that meet the intended purpose may be used. The body 725 may be affixed to the patient over the surgical opening, for example using surgical screws, surgical pins, adhesives or other suitable methods. In some examples, the guide 700 may instead be attached to the head holder 217.

The grip 710 may be provided on the body 725, for example as a plate or platform that is moveable with respect to the body 725. In some examples, the grip 710 may be a separate piece from the body 725 and may be assembled onto the body 725 after the body 725 has been affixed to the patient over the surgical opening. In the example shown, pins 714 provided on the grip 710 may be used to couple the grip 710 to the body 725 while still allowing the grip 710 to move relative to the body 725. The pins 714 may correspond to respective recesses or cut-outs 740 in the body 725, in order to provide for a limited range of motion for the grip 710 relative to the body 725. The grip 710 may be shaped to accommodate the curvature of the body 725.

The body 725 and the grip 710 may each define an opening through the guide 700, through which the access port 206 may be received. While the opening in the grip 710 may be sized for a tight fit with the access port 206, the opening in the body 725 may be sized to allow a limited range of motion for movement of the access port 206 (when in the grip 710). For example, the opening in the grip 710 may typically be a friction fit and no more than 1.1 times the size of the outer circumference of the access port 206, while the opening in the body 725 may be typically 1.5 times the size of the outer circumference of the access port 206.

The guide 700 may include one or more biasing members 745, such as elastic members or springs, that bias the grip 710 towards a centered or neutral position relative to the body 725. In the example shown, the biasing member(s) 745 may include one or more elastic members that extend between the corners of the body 725 and surround the grip 710. In some examples, the biasing member 745 may be a single elastic member that extends about the body 725. The biasing member(s) 745 may be in contact with the grip 710, for example the biasing member(s) 745 may be in contact against the pins 714. Other configurations may be suitable. For example, the biasing member(s) 745 may be in contact against the outer edge of the grip 710.

FIGS. 7B-7D illustrate how the guide 700 may allow a limited range of motion for the access port 206 received in the guide 700. FIGS. 7B-7D show a top-down view of the guide 700 with the access port 206 received in the grip 710. Dotted lines show the location of a surgical opening 702 over which the guide 700 is positioned and into which the access port 206 has been inserted.

FIG. 7B shows the grip 710 holding the access port 206 in a default centered position. In FIG. 7C, the grip 710 is moved by a displacing force D (e.g., exerted by a surgeon) to move the access port 206 off-center. As shown in FIG. 7C, this displacement causes stretching of the biasing member(s) 745. As a result, the biasing member(s) 745 exert biasing forces B, illustrated in FIG. 7D, which bias the grip 710 and the access port 206 held therein back to the centered position. The biasing forces B may enable return of the access port 206 to a default position (e.g., a centered position) with little or no exertion of forces onto the patient, in particular the patient's brain tissue. Such an arrangement may limit the range of motion of the access port 206 and may enable recentering of the access port 206, without the use of a separate articulated arm 219, which may help to simplify the surgical setup and/or provide greater access to the surgical opening.

In some examples, a locking mechanism, such as a cam lock (not shown) located on the side of the grip 710 may be used to lock the grip 710 at a position that is other than the default centered position with respect to the body 725. In some examples, the grip 710 may be attached to the body 725 in such a way (e.g., using pins 714 at different locations) such that the grip 710 is positioned off-center with respect to the body 725 by default (e.g., when no displacing force D is exerted). In some examples, the opening in the grip 710 may be off-center with respect to the body 725, such that the access port 206 held in the grip 710 is off-center with respect to the body 725. In some examples, the grip 710 may be interchangeable. For example, there may be different configurations for the grip 710, including configurations accommodating different centering points and/or different access port sizes.

In some examples, the two or more separate body members (not shown) may collectively form the body 725. For example, two or more body members may be attached to the patient in a distributed configuration around the surgical opening at a radius larger than that of the top portion of the access port 206. The biasing member(s) 745 may be bound by the body members and configured to contact the grip 710 (e.g., the pins 714 or the outer edge of the grip 710) such that the biasing member(s) 745 exert a restoring force on the grip 710 to bias the grip 710 towards the default position with respect to the body members.

In some examples, the grip 710 may have a side opening or may be openable to allow the access port 206 to be received into the grip 710, while the access port 206 is inserted into the surgical opening.

Although referred to as a grip, in some examples the grip 610, 620, 710 may not tightly grip the access port 206 and may instead loosely hold the access port 206 so that the access port 206 is enabled a certain freedom of movement relative to the grip 610, 620, 710. In some examples, such as described above, the grip 610, 620, 710 may be adjustable between tightly gripping the access port 206 (e.g., so that there is no movement of the access port 206 relative to the grip 610, 620, 710) and loosely holding the access port 206. The grip 610, 620, 710 may also be referred to as a grip holder or a holder.

In various example embodiments, the present disclosure provides a guide that may be used with an access port in port-based surgery. When the access port is held by the guide, the access port may be held in place in the patient, while still being moveable, to allow the surgeon to manipulate (e.g., using a hand or using tools inserted in the access port) the access port within a limited range of motion. The surgeon may also be able to lock the position and/or orientation of the access port with one hand, after changing the position and/or orientation of the access port. If not locked into place, the guide may serve to return the access port to a default (e.g., centered) position and/or orientation when the access port is no longer being manipulated. The returning force exerted by the guide onto the access port may also serve to limit the range of motion for manipulating the access port and may also serve to prevent the surgeon from manipulating the access port too far from the original default position and/or orientation.

The access port may be received into and locked into the guide while the access port is inserted into the surgical opening. Use of the guide may also enable the position and/or orientation of the access port to be tracked by a tracking system.

In some examples, one or more components of the guide may be constructed from a material that is at least partially transparent. This may provide the surgeon with visibility of the tissue beneath the guide, which may be useful while moving the access port. In some examples, the guide may be constructed using material compatible with one or more imaging modalities, including, but not limited to, MRI, PET, and CT.

While some embodiments or aspects of the present disclosure may be implemented in fully functioning computers and computer systems, other embodiments or aspects may be capable of being distributed as a computing product in a variety of forms and may be capable of being applied regardless of the particular type of machine or computer readable media used to actually effect the distribution.

At least some aspects disclosed may be embodied, at least in part, in software. That is, some disclosed techniques and methods may be carried out in a computer system or other data processing system in response to its processor, such as a microprocessor, executing sequences of instructions contained in a memory, such as ROM, volatile RAM, non-volatile memory, cache or a remote storage device.

A computer readable storage medium may be used to store software and data which when executed by a data processing system causes the system to perform various methods or techniques of the present disclosure. The executable software and data may be stored in various places including for example ROM, volatile RAM, non-volatile memory and/or cache. Portions of this software and/or data may be stored in any one of these storage devices.

The present disclosure describes example systems and methods in which a device may be intraoperatively configured based on the identification of a medical instrument. In some examples, one or more devices may be automatically controlled and/or configured by determining one or more context measures associated with a medical procedure. A “context measure”, as used herein, may refer to an identifier, data element, parameter or other form of information that may be relevant to the current state of a medical procedure. For example, a context measure may describe, identify, or be associated with the current phase or step of the medical procedure. In some examples, a context measure may identify the medical procedure, or the type of medical procedure, that is being performed. In some examples, a context measure may identify the presence of a tissue type during a medical procedure. In some examples, a context measure may identify the presence of one or more fluids, such as biological fluids or non-biological fluids (e.g. wash fluids) during the medical procedure, and may further identify the type of fluid. Each of these examples may relate to the image-based identification of information pertaining to the context of the medical procedure.

Examples of computer-readable storage media may include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

Furthermore, at least some of the methods described herein may be capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

At least some of the elements of the systems described herein may be implemented by software, or a combination of software and hardware. Elements of the system that are implemented via software may be written in a high-level procedural language such as object oriented programming or a scripting language. Accordingly, the program code may be written in C, C++, J++, or any other suitable programming language and may comprise modules or classes, as is known to those skilled in object oriented programming. At least some of the elements of the system that are implemented via software may be written in assembly language, machine language or firmware as needed. In either case, the program code can be stored on storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

While the teachings described herein are in conjunction with various embodiments for illustrative purposes, it is not intended that the teachings be limited to such embodiments. On the contrary, the teachings described and illustrated herein encompass various alternatives, modifications, and equivalents, without departing from the described embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

1. A guide for use with an access port for port-based surgery, comprising:
a body positionable over a surgical opening, the body comprising a linkage member for attachment to an external arm; and
a grip coupled to the body for removably receiving the access port into the surgical opening;
wherein at least one of the body and the grip is configured to restrict movement of the received access port to a limited range of motion;
wherein the linkage member has flexibility to enable a defined range of motion of the grip relative to the external arm; and
wherein the limited range of motion of the received access port is defined by the range of motion of the grip relative to the external arm.

2. The guide of claim 1 wherein the body is attachable to the external arm for positioning the guide over the surgical opening.

3. (canceled)

4. The guide of claim 1 wherein the linkage member is resilient to bias the grip to a default position and orientation relative to the external arm.

5. The guide of claim 1 wherein the flexibility of the linkage member is manually adjustable between at least some degree of flexibility and no flexibility.

6. The guide of claim 1 wherein the grip comprises a clamping member adjustable between a fully unlocked configuration and a fully locked configuration, wherein the fully unlocked configuration enables receipt of the access port into and removal of the access port from the grip, and the fully locked configuration fixes the position and orientation of the received access port within the grip.

7. The guide of claim 6 wherein the clamping is adjustable to a partially locked configuration in which angular movement of the received access port is restricted and movement of the received access port along a longitudinal axis of the access port is permitted.

8. The guide of claim 6 wherein the clamping member comprises a first portion attached to the body and a second portion, the second portion being moveable relative to the first portion to adjust the clamping member between the fully unlocked configuration and the fully locked configuration.

9. The guide of claim 6 wherein the body comprises a manually adjustable locking mechanism for adjusting the clamping member between the fully unlocked configuration and the fully locked configuration.

10. The guide of claim 9 wherein the locking mechanism is a manually rotatable collar positioned about the clamping member.

11. The guide of claim 1 wherein the body is affixable over the surgical opening and the grip has a defined range of motion relative to the body, the limited range of motion of the received access port further being defined by the range of motion of the grip relative to the body.

12. The guide of claim 11 further comprising one or more biasing members coupled to the body for biasing the grip towards a predefined default position with respect to the body.

13. The guide of claim 12 wherein the one or more biasing members comprise an elastic member coupled to the body and positioned about the grip, the elastic member engaging the grip at at least one point on the grip.

14. The guide of claim 12 further comprising a clasp mechanism for temporarily fixing the grip at a position other than the default position with respect to the body.

15. The guide of claim 11 wherein the grip is removably coupled to the body, the grip being interchangeable.

16. The guide of claim 1 further comprising one or more fiducial markers trackable by a tracking system, the one or more fiducial markers being provided in a predefined configuration on at least one of the body and the grip.

17. The guide of claim 16 wherein the body comprises a tracking arm extending from the body of the guide, the tracking arm including a plurality of branches of unequal length, and the fiducial markers are distributed among the branches.

18. A system for port-based surgery, comprising:
a guide for use with an access port for port-based surgery, the guide comprising:
a body positionable over a surgical opening, the body comprising a linkage member for attachment to an external arm; and
a grip coupled to the body for removably receiving the access port into the surgical opening;
wherein at least one of the body and the grip is configured to restrict movement of the received access port to a limited range of motion;
wherein the linkage member has flexibility to enable a defined range of motion of the grip relative to the external arm; and
wherein the limited range of motion of the received access port is defined by the range of motion of the grip relative to the external arm; and
the access port for insertion into the surgical opening, the access port being receivable by the guide.

19. The system of claim 18 further comprising:
one or more fiducial markers provided on at least one of the guide and the access port, the fiducial markers having a known position and orientation relative to the position and orientation of the access port; and
a tracking system for tracking the fiducial markers and determining the position and orientation of the access port based on the tracked fiducial markers.

20. The system of claim 18 further comprising:
an articulated arm to which the guide is attachable at a known position and orientation relative to the articulated arm; and
a tracking system for tracking the position and orientation of the articulated arm and determining the position and orientation of the access port based on the tracked position and orientation of the articulated arm.
